(19) United States
(12) Patent Application Publication
(10) Pub. No.: US 2012/0135112 A1
(43) Pub. Date: May 31, 2012

(54) YEAST, PREPARATION METHOD, COMPOSITION, APPARATUS AND USES THEREOF

(75) Inventors: Amelie Hiolle, Amfroipret (FR); Christian Lenoir, Marquette Lez Lille (FR); Jean-Charles Bartolucci, Saint-Andre (FR)

(73) Assignee: LESAFFRE ET COMPAGNIE, Paris (FR)

(21) Appl. No.: 13/377,912

(22) PCT Filed: Jun. 17, 2010
(86) PCT No.: PCT/FR2010/000451

§ 371 Date: Jan. 9, 2012

(30) Foreign Application Priority Data
Jun. 19, 2009 (FR) 09/2985

(51) Int. Cl. A23L 1/28; F26B 7/00; F26B 25/00; C12N 1/16

(52) U.S. Cl. 426/62; 435/255.1; 34/386; 34/236

(57) ABSTRACT

The invention relates to a method for preparing yeast, to a yeast obtained according to the method, to a food composition comprising the yeast, to an apparatus intended for implementing the method and to various uses of the yeast in a baker's dough. More specifically, the method consists in dehydrating yeast cream, granulating this yeast, and then drying.

The invention relates to a method for preparing yeast, to a yeast obtained according to the method, to a composition comprising the yeast, to an apparatus intended for implementing the preparation method and to various uses of the yeast.

A method for preparing active dry microorganisms, such as baker's yeast, having a dry matter content of between 90 and 98% by weight is already known from document WO 81/01415. This dry yeast is obtained by drying a mass of yeast on a fluidized bed, and then optionally in a drum dryer. The average size of the yeast particles obtained at the end of this method is between approximately 0.4 and 1.5 mm.

However, the use of a fluidized bed in the drying step does not make it possible to obtain yeast particles with a smooth surface. They therefore remain porous and exhibit a worse stability over time when they are brought into contact with air.

Moreover, document JP 55162928 describes a method for drying baker's yeast. This method consists in drying the yeast cream by passing it, rapidly and continuously, through a heat exchanger and then into a drum dryer so as to obtain a yeast having a dry matter content of at least 95% by weight.

The preparation of dry yeast, in granule form, by drying using one or two drum dryers is also known from document JP 05097687.

However, such drying techniques do not make it possible to obtain particles of dry yeast having a high fermenting capacity for several months while at the same time having a low moisture uptake.

In addition, yeast is generally known to be particularly sensitive to variations in temperature and in hygrometry related to storage conditions, and also to the presence of oxygen.

Thus, there remains a need to provide a method for producing dry yeast which is simple and rapid to implement, said method making it possible to smooth the surface of the yeast particles, thus ensuring better stability of the yeast over time with regard to maintaining its fermenting capacity and its low rate of moisture uptake.

An object of the invention is therefore a method for producing dry yeast. This method is characterized in that it consists in dehydrating a volume of yeast cream comprising sodium chloride-based brine, granulating the dehydrated yeast in order to extrude it, and then drying the extruded yeast so as to obtain a dry yeast having a dry matter content greater than or equal to 92% by weight.

This method has the advantage of providing a yeast which remains relatively insensitive to variations in temperature and in hygrometry, and to the presence of oxygen, which has a high fermenting capacity and a slow rate of moisture uptake, which remains stable for several months, or even a year, on contact with air when it is mixed with food ingredients, such as flour having, for example, a moisture content of from 7 to 15%, and which disperses correctly when it is incorporated into dough.

Another object of the invention is a dry yeast obtained according to the method described above, which is characterized in that it has an average diameter of between approximately 0.4 and 0.7 mm, in that it does not comprise any chemical additive, in that it is in spherule form, and in that it has a moisture uptake time of between approximately 1 and 2 hours.

The term “moisture uptake time” is intended to mean the time taken by the yeast to reach the moisture content of its environment, which can be, for example, a flour-based mix.

The yeast according to the invention can have a fermenting capacity greater than or equal to approximately 70 ml of gas given off over a period of at least six months at a temperature between approximately 15° and 25° C., when it is mixed with a flour having a moisture content of at least approximately 13%.

Preferably, the yeast according to the invention has an average diameter of between approximately 0.4 and 0.7 mm, and preferably of approximately 0.55 mm.

The yeast according to the invention has the advantage of having a homogeneous dry matter, and of dispersing easily and rapidly when it is incorporated into a mixture comprising flour and ingredients, thanks in particular to its small size.

The presence of a protective peripheral thin layer, consisting essentially of dead cells, forming a barrier to oxygen, allows the yeast to simultaneously have a low porosity, thus limiting its moisture uptake rate compared with dry yeasts lacking such a protective layer, a good fermenting capacity, and better stability over time in comparison with other dry yeasts, in particular of its fermenting capacity.

The yeast according to the invention has the advantage of having a moisture uptake rate which is between that of the known active dry yeasts (called active yeasts) which have a larger diameter and a protective thin layer of dead cells, and that of the dry yeasts, which are also known (called instant yeasts) and have no protective layer. Thus, the moisture uptake of the yeast according to the invention is therefore faster than that of active dry yeasts which have a protective layer, but slower than that of instant dry yeasts.

This protective layer also makes it possible to protect the yeast against variations in temperature during its storage, which are capable of impairing its fermenting capacity and/or its viability.

Finally, another advantage of the yeast according to the invention is that, when it is intimately mixed with food ingredients, there is no phenomenon of settling out or agglomeration over time. This is because it disperses correctly, in particular in the flour-based foods.

This is due to the fact that it exhibits a small size and is surrounded by a peripheral protective layer.

A third object of the invention is a composition comprising yeast as mentioned above, at least one ingredient chosen from gluten, salt, improvers, leavens, various wheat or rye flours, and any mixture thereof, each ingredient having, individually or as a mixture, a moisture content of between approximately 1 and 16% by weight.

A fourth object of the invention is a composition comprising yeast as described above, which is in the form of a gel and/or stick and which does not comprise any excipient.

An object of the invention is also an apparatus intended for implementing the present method. This apparatus is characterized in that it comprises at least one rotary filter, at least one granulator, at least one drum dryer, and at least one drying tower, each of these components being connected via feed pipes.

Another object of the invention is the use of the yeast in a composition intended for preparing a sweetened or unsweetened baker's dough.

The yeast according to the invention may optionally be of osmotolerant type, i.e. able to retain an advantageous fermenting capacity when it is incorporated into a sweetened dough.

Finally, the last objects of the invention concern uses of the yeast, as defined above, in an animal feed composition, or else in brewing and/or in enology.

The dehydration of a volume of yeast cream is preferably carried out under an air vacuum. This dehydration step leads to a yeast paste having approximately from 30% to 35% by weight of dry matter from a c yeast ream having, initially, approximately 16% by weight of dry matter.

At the end of the dehydration step, the yeast in the form of a paste is preferably subsequently extruded by means of a granulation step which defines the final particle size of the yeast.

The drying is preferably carried out batchwise, and consists, during a first step, in passing a stream of hot air through the volume of extruded yeast, and then, during a second step, in sending hot air under the lower part of the volume of partially dried yeast.

Preferably, the first step lasts approximately 6 hours, and the second approximately 2 hours.

During the first step, the stream of hot air preferably has a temperature of between approximately 35° and 38° C., and a flow rate of between approximately 20 000 and 30 000 m³/h. At the end of this first drying step, the dry matter content of the yeast is preferably approximately 90% by weight.

During the second drying step, the stream of hot air preferably has a temperature of between approximately 41° and 60° C., and a flow rate of between approximately 2700 m³/h and 4600 m³/h depending on the tonnage of dry yeast. On leaving this second drying step, the yeast, which is in the form of rather spherical particles, has a dry matter content of preferably between approximately 92% and 95% by weight.

In contrast to the techniques known up until now, the preparation method according to the invention does not use any additive during the drying.

The yeast resulting from this preparation method can be incorporated into a food composition, intended for the production of a sweetened or unsweetened baker's dough, in an amount of between approximately 0.40 and 99.5% by weight, relative to the total weight of the composition.

The invention will now be described in greater detail, by means of the figures which follow and which are given only by way of illustration, and also by means of examples of a food composition comprising the yeast according to the invention.

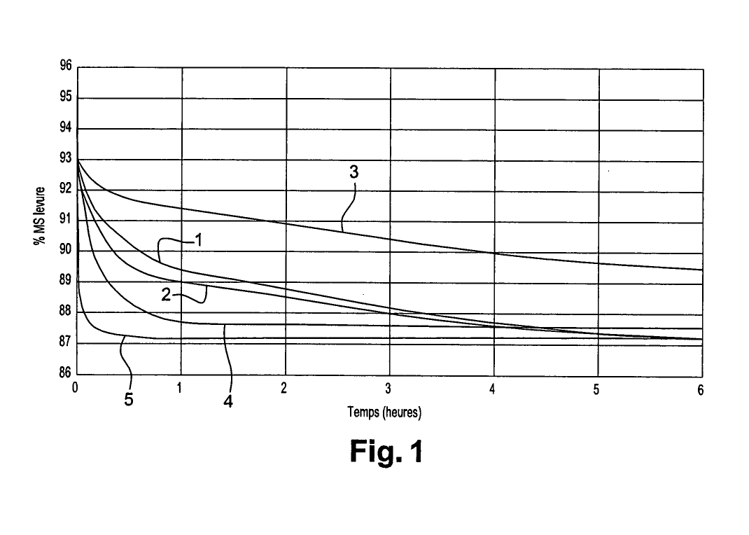

FIG. 1 represents the change in percentage of dry matter of a yeast according to the invention as a function of time, when it is incorporated into a flour which is not oven-dried, at a temperature of approximately 20° C., compared with that of the prior art yeasts.

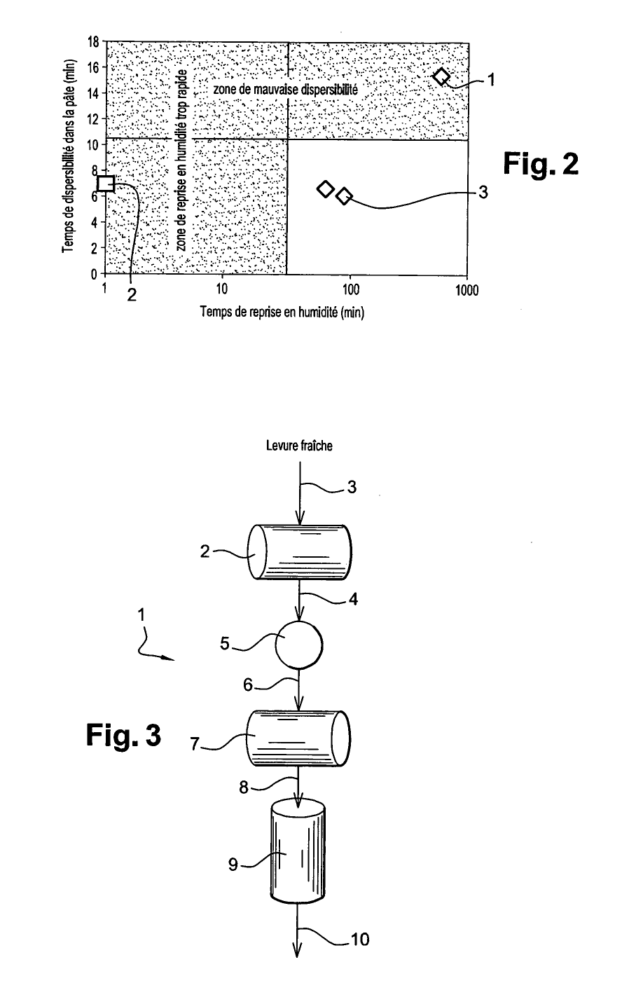

FIG. 2 represents the dispersibility time of the yeast in a dough as a function of moisture uptake time in a mix in order to reach 89% dry matter for yeasts of the invention and prior art yeasts.

FIG. 3 represents the apparatus of general reference 1, according to the invention, intended for implementing the yeast preparation method.

The change in percentage of dry matter of dry yeast as a function of time, which reflects its moisture uptake (FIG. 1), is obtained by applying the following procedure which uses an intimate mixing of flour and yeast.

Half the total amount of flour is placed in a jar. The dry yeast is added thereto, and is then covered with the other half of flour. The whole is mixed, the jar is closed and then placed in a temperature-controlled environment.

The storage time is counted from the moment when the yeast is brought into contact with the flour.

When the storage time has elapsed, the jar is opened, and its content is poured through a 250-micron sieve of an automatic sieve shaker (of Bioblock AS200 control g type).

At the end of the sieving, the yeast present in the sieve oversize is removed, and its percentage of dry matter is determined by means of the following mathematical calculation:

weight measured after an overnight period at 105° C. relative to the weight before stoving.

The various results obtained are collated on the curve of FIG. 1.

As presented in FIG. 1, reference curves 1 and 2 show the change in percentage of dry matter of the dry yeast according to the invention (average diameter between 0.5 and 0.6 mm).

Reference curve 3 shows the change in percentage of dry matter of a dry yeast of the prior art (called active yeast) having a higher average diameter (approximately 1.8 mm), and also having a protective peripheral layer of dead cells.

Reference curves 4 and 5 show the change in percentage of dry matter of a dry yeast of the prior art (called instant yeast) which is in the form of fine particles and which has no protective peripheral layer of dead cells.

From these curves, it can be noted that the behavior of the yeast of the invention (references 1 and 2) lies between that of the two types of yeast of the prior art (references 3, 4 and 5). It takes the yeast according to the invention between approximately 1 hour 10 min (reference 2) and 1 hour 40 min (reference 1) to reach a percentage of 89% dry matter, at 20° C.

On the other hand, it take reference yeast 3 approximately 10 hours to reach the same percentage of dry matter, and reference yeasts 4 and 5, 18 minutes and 1 minute, respectively.

Consequently, the yeasts according to the prior art either have a moisture uptake rate which is very slow but associated with an incapacity to disperse in the dough without prior rehydration (reference 3—yeast in spherule form with a large diameter of approximately 1.8 mm, often known as active dry yeast), or a moisture uptake rate which is very rapid due to the absence of a protective layer (references 4 and 5—instant dry yeasts).

The yeast according to the invention (references 1 and 2) has the advantage of combining a medium moisture uptake rate thanks to the presence of the protective layer, while at the same time having a satisfactory dispersibility in the dough owing to its small size.

As shown in FIG. 2, the active dry yeasts 1, of the prior art, with an average diameter of approximately 1.8 mm, have a long moisture uptake time and a long dispersibility time.

The instant dry yeasts 2 have a moisture uptake time of virtually zero and a satisfactory dispersibility time.

On the other hand, the yeasts 3 according to the invention have both a satisfactory moisture uptake time and a satisfactory dispersibility time. They exhibit a good balance between the constraints of dispersibility in the dough and the moisture uptake time in a flour mix.

The prior art yeasts 1 and 2 lie within zones of poor dispersibility and of moisture uptake that is too rapid.

As shown in FIG. 3, the apparatus comprises at least one dehydrating device consisting of a rotary filter 2, fed with fresh yeast via a pipe 3, and connected via a pipe 4 to a granulator 5, itself connected via a pipe 6 to a drum dryer 7. The drum dryer 7 is connected via a pipe 8 to a drying tower 9. The latter is connected to an outlet pipe 10 for the dry yeast.

The rotary filter 2 comprises a layer of starch on its external surface, and rotates at a speed of approximately 18 rpm. The surface of the filter 2 is regularly covered with starch.

The granulator 5 consists of a first cylinder with mixing arms which stir the dehydrated yeast and push it toward a second cylinder, at the end of which is a mesh or a perforated plate which can have openings of between 0.5 and 0.8 mm depending on the particle size desired for the yeast particles.

The drum dryer 7 consists of a rotary cylinder fitted with blades intended for stirring the yeast, a stream of hot air passing inside said cylinder. Its rotational speed is approximately 1 rpm.

The drying tower 9 consists of a cylinder in which there is a perforated plate. The yeast is placed on the plate and hot air is sent from below, thus creating a turbulence of the yeast and final drying thereof.

The apparatus operates in the following way.

A mixture consisting of fresh yeast at 16% by weight of dry matter and of brine comprising sodium chloride up until a conductivity of the mixture of between approximately 15 000 μS and 20 000 μS is reached, is sent, by means of the pipe 2a, to the rotary filter 2, the surface of which is pre-covered with a layer of starch.

On leaving the rotary filter, the yeast is dehydrated, and has a dry matter content of approximately 30% to 35% by weight.

The dehydrated yeast then enters the granulator 5 in order to be extruded therein according to a chosen particle size.

The extruded yeast is then directed, by virtue of the pipe 6, to the drum dryer 7 in which it remains for approximately 6 hours. The hot air which goes into the dryer 7 does not undergo any prior treatment. The temperature in the dryer 7 increases slowly and gradually so as to go from approximately 35° to approximately 38° C. over the course of 6 hours of drying.

On leaving the dryer 7, the yeast is directed toward the tower 9 in which hot air is sent to the top, thus creating a turbulence of the yeast, and therefore final drying thereof. The temperature in the tower 9 is approximately 42.5° C. throughout the drying period, which lasts approximately from 1 to 3 hours.

On leaving the tower 9, the dehydrated and dried yeast is then directed toward a storage silo.

According to known baking techniques, the food compositions, which illustrate the invention and are the subject of the examples below, are commonly called mixes, premixes or blends.

It is recalled that the blends consist of a mixture of yeast, of flour correctors and of bread improvers. The premixes consist of blends to which special flours, salt, sugar are added. The mixes comprise premixes to which the flour is added.

### EXAMPLE 1

### Food Composition Intended for Preparing a French Bread

| | Minimum | Maximum |
|---|---|---|
| Wheat flour type T45 | 96.92 | 80.66 |
| and/or T55 and/or T65 and/or T80 | | |
| Yeast 0.6 mm | 0.48 | 2.42 |
| Gluten | 0.09 | 1.61 |
| Fine salt | 1.45 | 2.01 |
| Dry wheat or rye leaven | 0.97 | 12.09 |
| Improver | 0.09 | 1.21 |
| Total | 100 | 100 |

The amounts are given in percentage by weight relative to the total weight of the composition.

The improver comprises various ingredients, such as ascorbic acid (from 10 to 100 ppm—preferably from 20 to 50 ppm), fungal alpha-amylase (from 0.5 to 15 ppm—preferably from 3 to 10 ppm), hemicellulases (from 5 to 150 ppm—preferably from 15 to 80 ppm), soy flour (from 0.05 to 0.5%—preferably from 0.1 to 0.3%), emulsifiers (DATEM from 0.05 to 0.5%—preferably from 0.1 to 0.3%, and/or mono- and diglyceride from 0.05 to 0.5%—preferably from 0.1 to 0.3 and/or SSL from 0.05 to 0.5%—preferably from 0.1 to 0.3%), deactivated yeast (from 0.05 to 0.15%).

This mixed composition, to which water is added, can be used in a breadmaking machine, or by manual kneading or kneading with a spatula.

The user of this composition will have to add approximately 60 to 62 ml of warm water per 100 g of composition.

### EXAMPLE 2

### Food Composition Intended for Preparing a Brioche Loaf

| | Minimum | Maximum |
|---|---|---|
| Wheat flour type T45 | 95.08 | 78.75 |
| and/or T55 and/or T65 and/or T80 | | |
| Yeast 0.6 mm | 0.47 | 2.36 |
| Gluten | 0.09 | 1.57 |
| Fine salt | 1.42 | 1.97 |
| Powdered sugar | 1.90 | 11.02 |
| Milk powder | 0.95 | 3.15 |
| Improver | 0.09 | 1.18 |
| Total | 100 | 100 |

The amounts are given as percentage by weight relative to the total weight of the composition.

The improver comprises various ingredients, such as ascorbic acid (from 10 to 100 ppm—preferably from 20 to 50 ppm), fungal alpha-amylase (from 0.5 to 15 ppm—preferably from 3 to 10 ppm), hemicellulases (from 5 to 150 ppm—preferably from 15 to 80 ppm), soy flour (from 0.05 to 0.5%—preferably from 0.1 to 0.3%), emulsifiers (DATEM from 0.05 to 0.5%—preferably from 0.1 to 0.3%, and/or mono- and diglyceride from 0.05 to 0.5%—preferably from 0.1 to 0.3, and/or SSL from 0.05 to 0.5%—preferably from 0.1 to 0.3%), deactivated yeast (from 0.05 to 0.15%).

This mixed composition, to which approximately 55 ml of warm water and 8 g of butter or margarine are added per 100 g of food composition, can be used in a breadmaking machine, or by manual kneading or kneading with a spatula.

### EXAMPLE 3

### Food Composition Intended for Preparing a Farmhouse Bread

| | Minimum | Maximum |
|---|---|---|
| Wheat flour type 55 and/or | 85.189 | 69.992 |
| type 65 | | |
| Rye flour T130 | 9.68 | 13.124 |
| Whole wheat flour | 1.936 | 4.375 |
| Malted wheat flour | 0.097 | 0.262 |
| Dry wheat | 0.968 | 4.374 |
| and/or rye leaven | | |
| Yeast 0.6 mm | 0.484 | 2.625 |
| Gluten | 0.097 | 1.749 |
| Fine salt | 1.452 | 2.187 |
| Improver | 0.097 | 1.312 |
| Total | 100.00 | 100.00 |

The amounts are given as percentage by weight relative to the total weight of the composition.

The improver comprises various ingredients, such as ascorbic acid (from 10 to 100 ppm—preferably from 20 to 50 ppm), fungal alpha-amylase (from 0.5 to 15 ppm—preferably from 3 to 10 ppm), hemicellulases (from 5 to 150 ppm—preferably from 15 to 80 ppm), soy flour (from 0.05 to 0.5%—preferably from 0.1 to 0.3%), emulsifiers (DATEM from 0.05 to 0.5%—preferably from 0.1 to 0.3%, and/or mono- and diglyceride from 0.05 to 0.5%—preferably from 0.1 to 0.3, and/or SSL from 0.05 to 0.5%—preferably from 0.1 to 0.3%), deactivated yeast (from 0.05 to 0.15%).

This mixed composition, to which approximately 62 to 65 ml of warm water are added per 100 g of food composition, can be used in a breadmaking machine, or by manual kneading or kneading with a spatula.

### EXAMPLE 4

### Food Composition Intended for Preparing a Multi-Grain and Cereal Bread

| | Minimum | Maximum |
|---|---|---|
| Wheat flour type 45 and/or 55 | 85.84 | 70.68 |
| and/or type 65 | | |
| Malted rye flakes | 5.15 | 8.48 |
| Sunflower seed | 1.72 | 4.24 |
| Brown linseed | 1.72 | 3.53 |
| Slivers of soy | 1.72 | 3.53 |
| Yeast 0.6 mm | 0.43 | 2.12 |
| Gluten | 0.85 | 2.83 |
| Fine salt | 1.29 | 1.41 |
| Dry wheat or rye leaven | 0.85 | 2.12 |
| Improver | 0.43 | 1.06 |
| Total | 100.00 | 100.00 |

The amounts are given as percentage by weight relative to the total weight of the composition.

The improver comprises various ingredients, such as ascorbic acid (from 10 to 100 ppm—preferably from 20 to 50 ppm), fungal alpha-amylase (from 0.5 to 15 ppm—preferably from 3 to 10 ppm), hemicellulases (from 5 to 150 ppm—preferably from 15 to 80 ppm), soy flour (from 0.05 to 0.5%—preferably from 0.1 to 0.3%), emulsifiers (DATEM from 0.05 to 0.5%—preferably from 0.1 to 0.3%, and/or mono- and diglyceride from 0.05 to 0.5%—preferably from 0.1 to 0.3, and/or SSL from 0.05 to 0.5%—preferably from 0.1 to 0.3%), deactivated yeast (from 0.05 to 0.15%).

This mixed composition, to which approximately 58 to 60 ml of warm water are added per 100 g of food composition, can be used in a breadmaking machine, or by manual kneading or kneading with a spatula.

### EXAMPLE 5

### Food Composition Intended for Preparing a Rye Bread

| | Minimum | Maximum |
|---|---|---|
| Wheat flour type 55 and/or | 76.55 | 44.44 |
| type 65 | | |
| Rye flour T70 and/or | 19.14 | 44.44 |
| T85 and/or T130 | | |
| Gluten | 0.96 | 2.67 |
| Fine salt | 1.43 | 1.78 |
| Yeast 0.6 mm | 0.48 | 2.67 |
| Dry wheat or rye leaven | 0.96 | 2.67 |
| Improver | 0.48 | 1.33 |
| Total | 100.00 | 100.00 |

The amounts are given as percentage by weight relative to the total weight of the composition.

The improver comprises various ingredients, such as ascorbic acid (from 10 to 100 ppm—preferably from 20 to 50 ppm), fungal alpha-amylase (from 0.5 to 15 ppm—preferably from 3 to 10 ppm), hemicellulases (from 5 to 150 ppm—preferably from 15 to 80 ppm), soy flour (from 0.05 to 0.5%—preferably from 0.1 to 0.3%), emulsifiers (DATEM from 0.05 to 0.5%—preferably from 0.1 to 0.3%, and/or mono- and diglyceride from 0.05 to 0.5%—preferably from 0.1 to 0.3, and/or SSL from 0.05 to 0.5%—preferably from 0.1 to 0.3%), deactivated yeast (from 0.05 to 0.15%).

This mixed composition, to which approximately 56 to 58 ml of warm water are added per 100 g of food composition, can be used in a breadmaking machine, or by manual kneading or kneading with a spatula.

### EXAMPLE 6

### Food Composition Intended for Preparing a Bran Bread

| | Minimum | Maximum |
|---|---|---|
| Wheat flour type 55 and/or | 86.12 | 71.11 |
| type 65 | | |
| Wheat bran | 9.57 | 17.77 |
| Gluten | 0.96 | 2.67 |
| Fine salt | 1.43 | 1.78 |
| Yeast 0.6 mm | 0.48 | 2.67 |
| Dry wheat or rye leaven | 0.96 | 2.67 |
| Improver | 0.48 | 1.33 |
| Total | 100.00 | 100.00 |

The amounts are given as percentage by weight relative to the total weight of the composition.

The improver comprises various ingredients, such as ascorbic acid (from 10 to 100 ppm—preferably from 20 to 50 ppm), fungal alpha-amylase (from 0.5 to 15 ppm—preferably from 3 to 10 ppm), hemicellulases (from 5 to 150 ppm—preferably from 15 to 80 ppm), soy flour (from 0.05 to 0.5%—preferably from 0.1 to 0.3%), emulsifiers (DATEM from 0.05 to 0.5%—preferably from 0.1 to 0.3%, and/or mono- and diglyceride from 0.05 to 0.5%—preferably from 0.1 to 0.3, and/or SSL from 0.05 to 0.5%—preferably from 0.1 to 0.3%), deactivated yeast (from 0.05 to 0.15%).

This mixed composition, to which approximately 60 to 62 ml of warm water are added per 100 g of food composition, can be used in a breadmaking machine, or by manual kneading or kneading with a spatula.

Example 7 which follows is a food composition illustrating the invention, commonly termed a premix.

### EXAMPLE 7

### Food Composition Intended for Preparing a Brioche Loaf

| | Minimum | Maximum |
|---|---|---|
| Wheat flour type 45 and/or | 82.67 | 10 |
| T55 and/or T65 | | |
| Yeast 0.6 mm | 1.67 | 10 |
| Gluten | 0.33 | 6.67 |
| Salt | 5 | 8.33 |
| Powdered sugar | 6.67 | 46.67 |
| Milk powder | 3.33 | 13.33 |
| Improver | 0.33 | 5 |
| Total | 100.00 | 100.00 |

The amounts are given as percentage by weight relative to the total weight of the composition.

The improver comprises various ingredients, such as ascorbic acid (from 10 to 100 ppm—preferably from 20 to 50 ppm), fungal alpha-amylase (from 0.5 to 15 ppm—preferably from 3 to 10 ppm), hemicellulases (from 5 to 150 ppm—preferably from 15 to 80 ppm), soy flour (from 0.05 to 0.5%—preferably from 0.1 to 0.3%), emulsifiers (DATEM from 0.05 to 0.5%—preferably from 0.1 to 0.3%, and/or mono- and diglyceride from 0.05 to 0.5%—preferably from 0.1 to 0.3, and/or SSL from 0.05 to 0.5%—preferably from 0.1 to 0.3%), deactivated yeast (from 0.05 to 0.15%).

This premix composition can be used for preparing a brioche loaf, by mixing 30 g of premix with 70 g of flour, to which approximately 55 ml of warm water and approximately 8 g of butter or margarine are added.

This premix composition can also be incorporated in a proportion ranging from 15 to 50% by weight, relative to the total weight of the composition.

This composition can also be used for preparing blends, by reducing or even eliminating the amount of flour and the dose for final use, and also for the mixes of Examples 1 to 6.

### EXAMPLE 8

### Composition of a Yeast of “Two-in-One” Type

| | Minimum | Maximum |
|---|---|---|
| Yeast 0.6 mm | 99.50 | 98.50 |
| Improver | 0.5 | 1.50 |
| Total | 100.00 | 100.00 |

The improver comprises various ingredients, such as ascorbic acid (from 10 to 100 ppm—preferably from 20 to 50 ppm), fungal alpha-amylase (from 0.5 to 15 ppm—preferably from 3 to 10 ppm), hemicellulases (from 5 to 150 ppm—preferably from 15 to 80 ppm), soy flour (from 0.05 to 0.5%—preferably from 0.1 to 0.3%), emulsifiers (DATEM from 0.05 to 0.5%—preferably from 0.1 to 0.3%, and/or mono- and diglyceride from 0.05 to 0.5%—preferably from 0.1 to 0.3, and/or SSL from 0.05 to 0.5%—preferably from 0.1 to 0.3%), deactivated yeast (from 0.05 to 0.15%).

Comparative Stability Test

The stability of the fermenting capacity of the yeast according to the invention stored in a flour mix was tested according to an accelerated laboratory test, and compared with other known dry yeasts.

This test consists in mixing the flour with the yeast so as to obtain a mix, in storing this mixture at a temperature of approximately 30° C., and in evaluating the loss of fermentative strength relative to the initial state, i.e. without storage. This loss of fermentative strength is obtained by measuring the CO₂ given off by means of a fermentometer, before storage and after 14 days of storage of the mix at approximately 30° C.

This test makes it possible to study the behavior of the yeast of the invention when it is subjected both to hydric stress and to oxidative stress.

The flour is used with a moisture content of approximately 14.5%.

Tables I and II hereinafter collate the results obtained when the yeast is, respectively, used in normal dough (Table I) and in sweetened dough (Table II).

TABLE I
(normal dough)

| | Stability |
|---|---|
| Dry yeast sold under the trademark “Aigle du | from 2 to 14% depending |
| Nord” or “Saf Instant Rouge” | on the origin |
| Dry yeast sold under the trademark “Yeast in | from 22 to 25% |
| classic” | |
| Dry yeast sold under the trademark “High | from 1 to 15% |
| Power Plus” | |
| Yeast of the invention | from 44 to 49% |

TABLE II
(sweetened dough)

| | Stability |
|---|---|
| Dry yeast sold under the trademark “Bruggeman” | from 19 to 29% |
| Dry yeast sold under the trademark “Saf Instant Or” | from 39 to 48% |
| Dry yeast sold under the trademark “Saf-mix-SPM | 49% |
| Gold” | |
| Yeast of the invention | from 61 to 64% |

The results of these studies clearly show that the yeast of the invention exhibits a better stability of the fermenting capacity, in a mix, when it is used in a normal dough and in a sweetened dough.

Comparative Dispersion Test

This test consists in evaluating the dispersion of the yeast of the invention in comparison with another known dry yeast, when it is incorporated into a flour-based animal feed.

This test consists in incorporating a yeast sample into a food intake and then mixing the whole together.

The food intake comprises 33% of wheat, 33% of soy flour and 34% of rapeseed flour. The yeast is added in an amount of 0.1% relative to the total weight of the feed intake.

The dispersion capacity of the yeast is obtained by calculating the coefficient of variation (CV) according to the references of the Centre Technique de l'Alimentation Animale [Technical Centre for Animal Feed] of the company TECALIMAN.

A coefficient of variation value of less than 10% indicates good dispersion of the ingredient added to the food intake; and a coefficient value of less than 5% indicates optimum dispersion.

The results are collated in Table III hereinafter.

TABLE III

| | | | % coefficient |
|---|---|---|---|
| | | Desired amount | of variation |
| | % addition | (CFU/g) | (CV) |
| Control dry yeast (diameter: | 0.1 | 8 × 10⁶ | 2.4 |
| 1.8 mm) sold under the | | | |
| trademark “Actisaf” | | | |
| Yeast according to the | 0.1 | 7.3 × 10⁶ | 1.1 |
| invention (diameter: 0.5 mm) | | | |
| Yeast according to the | 0.1 | 9 × 10⁶ | 1.1 |
| invention (diameter: 0.6 mm) | | | |

The results show that the yeast according to the invention exhibits excellent dispersion when it is incorporated into a flour-based food.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. Method for producing dry yeast comprising steps of:
dehydrating a volume of yeast cream comprising a sodium chloride-based brine so as to obtain dehydrated yeast,
granulating the dehydrated yeast in order to extrude it and obtain extruded yeast, and
drying the extruded yeast so as to obtain a dry yeast having a dry matter content greater than or equal to 92% by weight.

18. The method according to claim 17, wherein the drying is carried out batchwise, and consists in:
in a first drying step, passing a stream of hot air through the volume of yeast, and
in a second drying step, sending hot air under the lower part of the volume of yeast.

19. The method according to claim 18, wherein the first drying step lasts approximately 6 hours, and the second drying step lasts approximately 2 hours.

20. The method according to claim 18, wherein in the first drying step, the stream of hot air has a temperature of between approximately 35° and 38° C., and in the second drying step, the stream of hot air has a temperature of between approximately 41° and 60° C.

21. The method according to claim 19, wherein in the first drying step, the stream of hot air has a temperature of between approximately 35° and 38° C., and in the second drying step, the stream of hot air has a temperature of between approximately 41° and 60° C.

22. The method according to claim 18, wherein in the first drying step, the stream of hot air has a flow rate of between approximately 20 000 and 30 000 m³/h, and in the second drying step, the stream of hot air has a flow rate of approximately 2700 m³/h to 4600 m³/h.

23. The method according to claim 17, wherein the dehydration step is carried out under an air vacuum.

24. Dry yeast obtained by the method according to claim 17, wherein said dry yeast:
has an average diameter of between approximately 0.4 and 0.7 mm,
does not comprise any chemical additive,
is in spherule form, and
has a moisture uptake time of between approximately 1 and 2 hours.

25. The dry yeast according to claim 24, wherein said dry yeast has an average diameter of approximately 0.55 mm.

26. Composition comprising the dry yeast according to claim 24, at least one ingredient chosen from gluten, salt, improvers, leavens, various wheat or rye flours, and any mixture thereof, each ingredient having, individually or as a mixture, a moisture content of between approximately 1 and 16% by weight.

27. The composition according to claim 26, wherein the dry yeast is present in an amount of between approximately 0.40 and 99.9% by weight, relative to the total weight of the composition.

28. Apparatus intended for implementing the method according to claim 17, comprising at least one rotary filter, at least one granulator, at least one drum dryer, and at least one drying tower, each of these components being connected via feed pipes.

29. The apparatus according to claim 28, wherein:
the drum dryer is a rotary cylinder fitted with blades,
the granulator is a cylinder having mixing arms and an extrusion mesh with openings whose diameter is between 0.4 and 0.7 mm, and
the drying tower is a cylinder comprising a perforated plate.

30. A composition for preparing a sweetened or unsweetened baker's dough, the composition comprising the dry yeast according to claim 24.
